(19) United States
(12) Patent Application Publication
(10) Pub. No.: US 2026/0083476 A1
(43) Pub. Date: Mar. 26, 2026

(54) QUIET SURGICAL SUCTION

(71) Applicant: Daniel R. Jacobson, Chicago, IL (US)

(72) Inventor: Daniel R. Jacobson, Chicago, IL (US)

(21) Appl. No.: 19/338,328

(22) Filed: Sep. 24, 2025

Related U.S. Application Data
(60) Provisional application No. 63/698,044, filed on Sep. 24, 2024.

(51) Int. Cl. A61B 17/34

(52) U.S. Cl. CPC A61B 17/3421; A61B 17/3439; A61B 2017/3433

(57) ABSTRACT

Disclosed are devices, systems and methods for reducing noise from surgical and dental suction tips, especially Frazier and Fukushima type cannulas, by altering the size, shape, and contour of control valves. More specifically, the disclosed devices modify sound generated by traditional suction tips and/or thumbhole controls of various suction cannulas, (also called control valves, control vents, grip plates, and/or suction interrupters) by decreasing the intensity of the sound output of the suction cannula's amplitude and changing the frequency to a less annoying pitch.

### CROSS-REFERENCE TO RELATED APPLICATIONS

This application claims the benefit of U.S. Provisional Application No. 63/698,044 entitled “SURGICAL SUCTION DEVICES” filed Sep. 24, 2024, the disclosure of which is incorporated by reference herein in its entirety.

### FIELD OF THE INVENTION

The present subject matter relates generally to surgical suction devices, and more particularly to suction devices for regulating and controlling suction noise in an aspiration line, and which may be included in aspirators such as for example dentistry, surgery, or cosmetic tools. More specifically, the device of the present invention includes a body having an inlet port and an outlet port defining a chamber in the body, and a lateral inlet used as a thumb valve control, intersecting the chamber such that a main fluid stream is generated with reduced aerodynamic noise.

### BACKGROUND OF THE INVENTION

During surgical operations, it is often desirable to remove fluid such as blood and other body fluids from the body cavity being operated on. This is commonly done with an aspirator or suction device connecting a vacuum source to a nozzle having a hollow tip, and such devices are widely used in a plurality of professional fields and, namely, in the medical field. The suction aspirator device, often called a suction cannula, or suction tip, or suction catheter, or suction tube is used to remove blood, lymph, saline, water, saliva, and/or debris from a surgical or dental treatment area, such as a surgical site and/or from a patient's mouth during a dental treatment or surgery.

Noise in medical aspirators is recognized as a very significant problem. Indeed, it is a source of fatigue, irritation, and stress accumulation, and it represents a real risk of hearing acuity degradation for medical personnel. Furthermore, the operating room or surgical theater is a noisy environment. Loud noise from suction cannulas is but one unnecessary contributor to this accumulated noise. It would therefore be a significant advance in the art of controlled suction aspirating devices to provide a low-noise suction control device and an aspirator using such a device, which desirably generates low and/or minimally annoying noise, according to preferred structures as contemplated in the present invention. It would also be desirable to provide an aspirator which enhances the physical comfort of a user through an ergonomic design providing comfort, weight balance and/or simply operation.

Accordingly, there is need for further improvement in surgical instruments and tools, and the present subject matter is such improvement.

### BRIEF SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

Excess noise generated by standard surgical suction devices is based on the effects of aerodynamic noise theory concerning pipe geometries. An acoustic source that is generated by variations in velocity and pressures would create a dipole whistle (Reethof 1978). Current devices on the market allow eddy currents or turbulence to form, resulting in acoustic fluctuations at high intensity levels, which can create noise pollution. The present disclosure addresses noise generation by incorporating altered tip and control valve geometries of the suction device, including in some embodiments the incorporation of multiple holes around the edge of a suction nozzle tip to dissipate some of the airflow generated by the negative pressure of the system, which desirably provides alternative air outlets for air to escape. thereby reducing noise.

The present invention relates to a noise reducing aspirator which desirably lowers a sound level and/or improves the sound characteristics, while maintaining or improving suction performance. In accordance with various teachings of the present invention, there is disclosed a suction device for an aspirator connectable to a vacuum source, the suction device including a body including an external surface, an inlet port located at a first end of the body, an outlet port located at a second end of the body, a chamber extending inside the body from the inlet port to the outlet port along a longitudinal axis, the chamber defining a flow direction from the inlet port to the outlet port, the suction device further including a bypass inlet diverging through the body from the external surface to the chamber generally along the flow direction.

The proposed solutions are significant improvements over prior art devices due to the significant reduction of noise pollution obtained herein. Various embodiments of the present invention incorporates a circular, tear-drop, elliptical and/or ovoid shaped hole on the body of the suction tip (frequently called a control valve, control vent, grip plate, and/or suction interrupter) where the surgeon can grip the device to provide greater control of the suction strength, by allowing the surgeon to progressively cover and/or uncover the hole with their finger.

The invention specifically optimizes the size, shape, and contour of the control vent and tip of the suction canula (also called a suction catheter) to reduce the noise created by airflow. In one prototype, the disclosed suction devices reduced the decibel level from 85 dB to 65 dB when compared to a commercially available and popularly used disposable Frazier tip suction having a round control vent.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings.

FIG. 1 depicts a perspective view of one exemplary embodiment of an aspirator tool constructed in accordance with the various teachings of the present invention;

FIG. 2A through 2F depict various views of one exemplary embodiment of a handle for an aspirator tool;

FIG. 3A depicts a perspective view of the handle of FIG. 2A;

FIG. 3B depicts a cross-section of the handle of FIG. 3A, taken along plane 3B-3B;

FIG. 3C depicts a cross-section of the handle of FIG. 3A, taken along plane 3C-3C;

FIG. 3D depicts various exemplary dimensions of the handle FIG. 2A;

FIGS. 4A through 4D depict various views of one alternative embodiment of a noise-reducing handle for an aspirator tool;

FIGS. 5A and 5B depict various views of another alternative embodiment of a noise-reducing handle for an aspirator tool;

FIGS. 6A through 6E depict various views of another alternative embodiment of a noise-reducing handle for an aspirator tool; and

FIG. 7 depicts views of additional alterative handle embodiments.

### DETAILED DESCRIPTION OF THE INVENTION

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals may represent similar parts throughout the several views of the drawings. In addition, the following is a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In various embodiments, the terms “including,” “comprising” and variations thereof, as used in this disclosure, should be interpreted as “including, but not limited to,” unless expressly specified otherwise. The terms “a,” “an,” and “the,” as used in this disclosure, mean “one or more,”unless expressly specified otherwise.

In some embodiments, devices and/or device components that may be disclosed in communication with each other need not necessarily be in continuous communication with each other, unless expressly specified otherwise. In addition, components that are in direct contact with each other may contact each other directly or indirectly through one or more intermediary articles or devices. The device(s) disclosed herein may comprise various surgical materials, including plastics, metals, silicone, rubber, latex, or any other biocompatible material, and/or any combination(s) thereof.

Although process steps, method steps, or the like, may be described in a sequential order, such processes and methods may be configured in alternate orders. In other words, any sequence or order of steps that may be described does not necessarily indicate a requirement that the steps be performed in that order. The steps of the processes or methods described herein may be performed in any order practical. Further, some steps may be performed simultaneously.

When a single component, device and/or article is described herein, it should be readily apparent that more than one component, device and/or article may be used in place of a single component, device and/or article, unless otherwise noted. Similarly, where more than one component, device and/or article is described herein, it should be readily apparent that a single component, device and/or article may be used in place of the more than one component, device and/or article, unless otherwise noted. The functionality or the features of a component, device and/or article may be alternatively embodied by one or more other components, devices and/or articles which are not explicitly described as having such functionality or features.

Certain exemplary embodiments will now be described to provide an overall understanding of the principles of the structure, function, manufacture, and use of the components, devices and methods disclosed herein. One or more examples of these embodiments are illustrated in the accompanying drawings. Those of ordinary skill in the art will understand that the components, devices and/or methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The sound output of medical and/or dental aspirators is often a result of air turbulence. The rapid oscillating motions within a turbulent air flow can cause the air surrounding the turbulent region to vibrate, which air vibration is perceived to the human ear as sound. Sound output can also be generated by the meeting of turbulent air streams flowing at different velocities, which is commonly referred to as a “shear layer.” At present, the use of surgical and dental suction is complicated by excess and irritating noise as negative pressure generates turbulent airflow and subsequent audible sound at the suction tube tip and at the vent holes if present. According to Federal Health Guidelines, the sound level of the average noise exposure should not exceed 85 dB. Studies to determine the noise of suction-only found that these approach these unsafe levels. Beyond the potential hearing impairments, noises within the operating room are shown to have negative effects on the fine motor skills, cognition, and mood of surgeons and staff. For example, subjects in a study to determine the effects of operating room noise showed decreased time to complete line tracking in the form of a stylus through a channeled maze while in silence. Also, these subjects demonstrated decreased delayed recall in silence thus validating that a surgeon's concentration and all OR personnel is hindered in a noise-filled environment.

The disclosed embodiments specifically optimize the size, shape, and contour of a control vent and tip of a suction canula (also called a suction catheter) to reduce the noise created by airflow. In one prototype, the disclosed suction tip and associated devices reduced the decibel level from 85 dB to 65 dB when compared to a commercially available and popularly used disposable Frazier tip suction having a round control vent.

Various embodiments of the present invention comprises an open-ended housing adapted to be held by a physician or surgical assistant, and having a first end which is connected to a source of suction, which may comprise any convenient suction source normally provided with medical and/or dental equipment, and a second end adapted to be connected to an aspirator tip for the reception of fluids and debris including blood, lymph, water, saline, etc. The second end will desirably have a diameter into which the aspirator tube/tip fits into securely. Both ends of the housing can be adapted for easy removal and replacement of the suction hose and/or aspirator tube/tip.

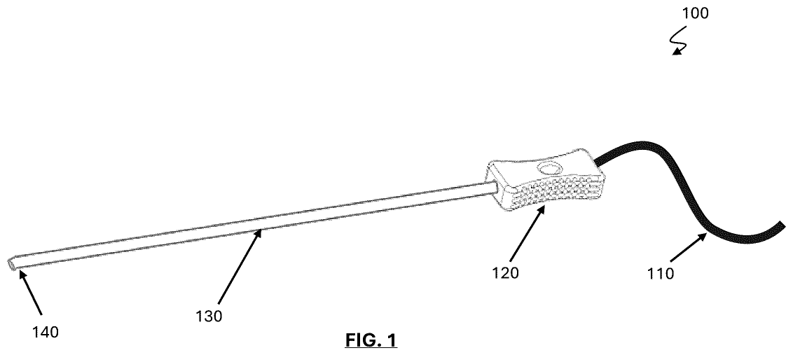

FIG. 1 depicts one exemplary embodiment of an aspirator tool 100 constructed in accordance with the various teachings of the present invention. In this embodiment, the tool includes a suction source tube 110, a handle 120 an aspiration tube 130 and a tube tip 140.

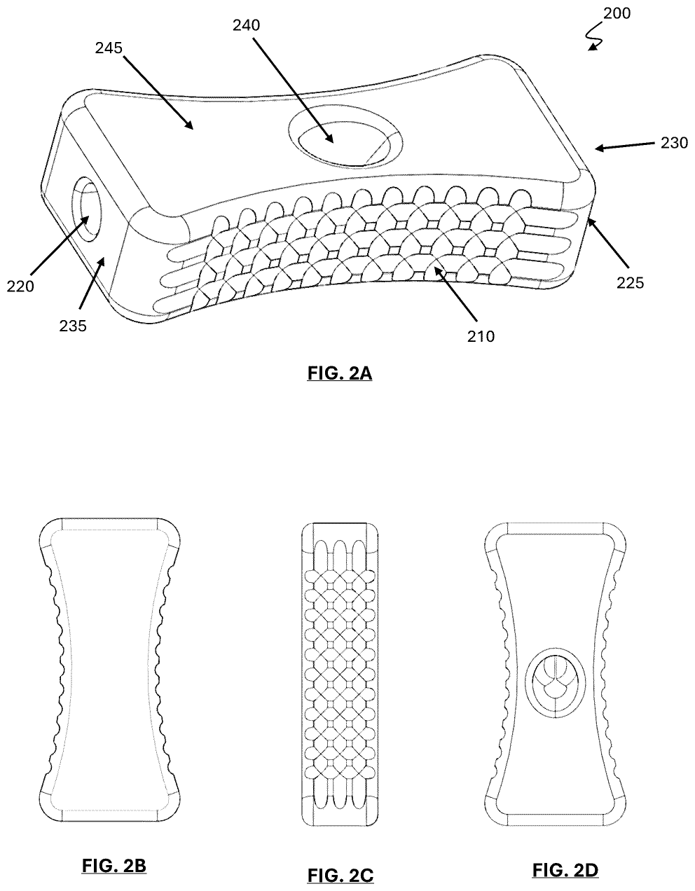

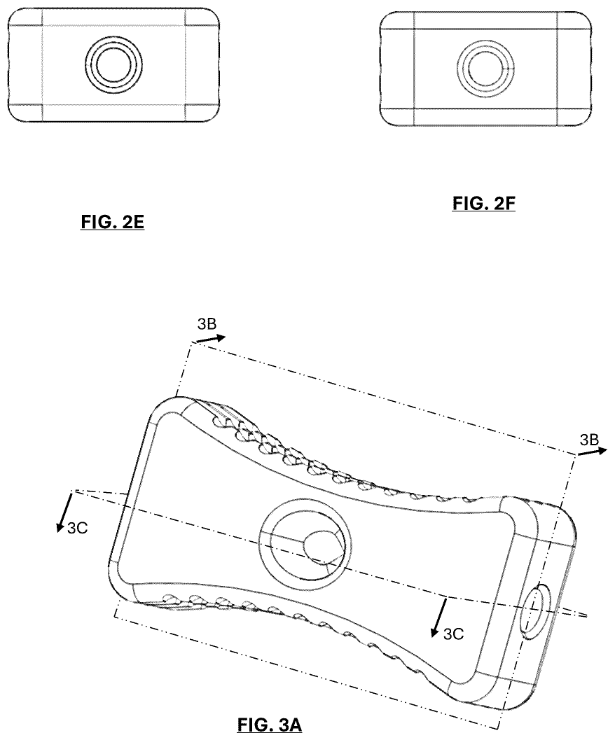

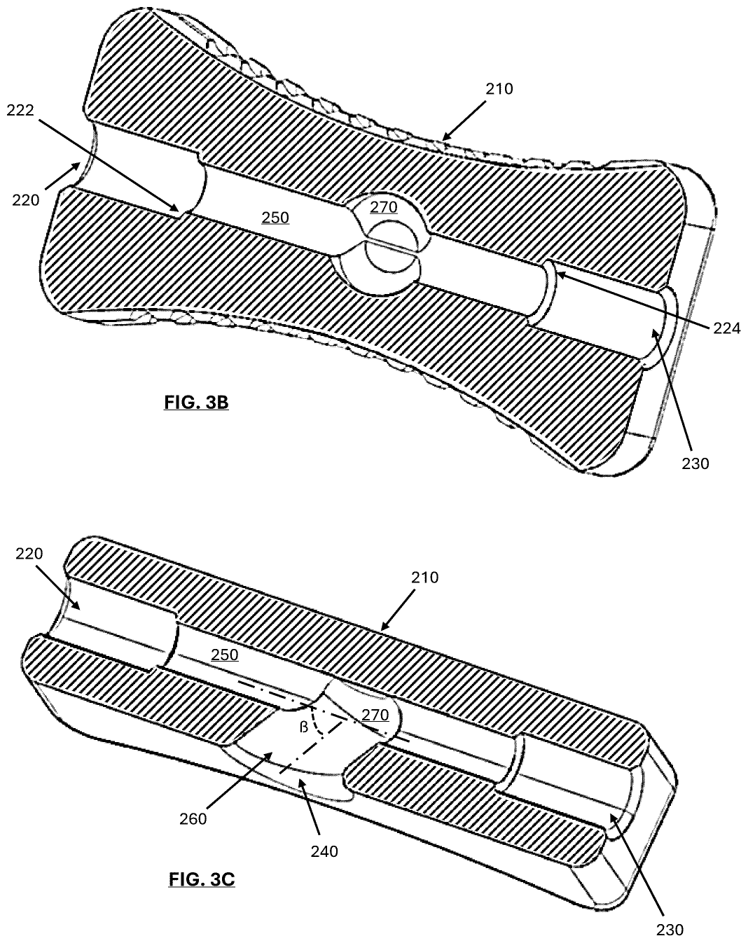

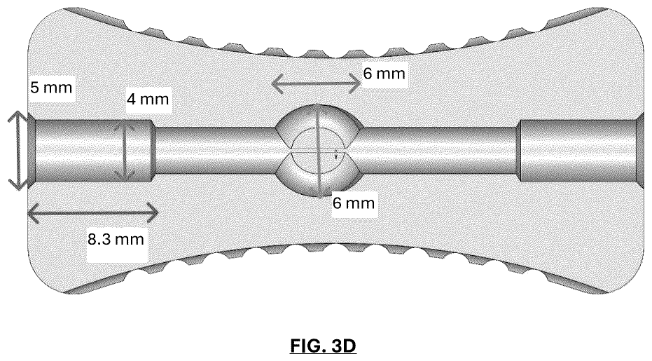

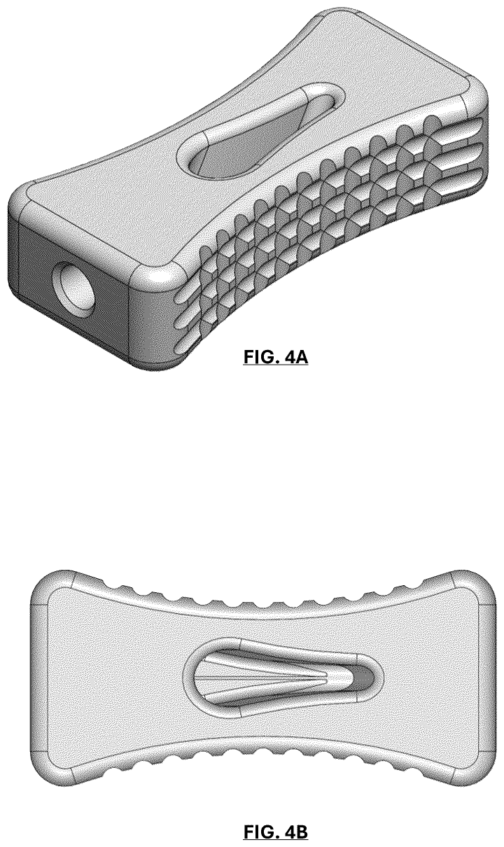

FIGS. 2A through 2F depict one exemplary embodiment of a handle 200 for an aspirator tool. The handle can include a handle body 210 having a suction inlet opening 220 at a proximal end 225, a suction outlet opening 230 at a distal end 235 and a secondary opening or control opening 240 on a top surface 245. As best seen in the cross-sectional view of FIG. 3B, a primary lumen 250 can desirably extend through the handle from the inlet opening 220 to the outlet opening 230, with a first necked-down portion 222 and a second necked-down portion 224 of the primary lumen depicted therein. As best seen in the cross-sectional view of FIG. 3C, a secondary lumen 260 can extend from the control opening 240 to meet with the lumen primary lumen 250 at an enlarged air chamber 270.

FIGS. 3A through 3C depict a perspective and 2 cross-sectional views of the handle of FIG. 2A.

As best seen in FIGS. 3B and 3C, the secondary lumen is in fluid communication with the primary lumen, with the two lumens each reducing in diameter at respective neck locations and eventually meeting at an air chamber 270. Desirably, the secondary lumen enters the enlarged air chamber at an angle ß relative to the suction inlet opening 220 (see FIG. 3C), with the openings and the lumens desirably smooth and/or chamfered for a smooth air flow therethrough. As best seen in FIG. 3C, the secondary lumen connects to the primary lumen (which may also be referred to as the main suction tube) via a slanted path. Which desirably permits outside air to merge with the air passing through in the main suction tube. This configuration can also release excessive suction and thereby prevent excessive suction at the aspirator tip, thereby reducing noise levels compared to prior art devices. In one exemplary embodiment which demonstrated superior sound reduction measurements, the secondary lumen can intersect the primary lumen at an obtuse angle of approximately 145 degrees. In various embodiments, angle ß can be an acute angle or angle ß can be an obtuse angle. Even if not specifically depicted in the various embodiments shown herein, it should be understood that the secondary stream may be depicted as angled towards the direction of the primary stream in some embodiments, while alternative embodiments may incorporate a the secondary stream which is angled away from the primary stream to the same or similar degree.

The disclosed embodiment desirably modifies the meeting point areas and/or velocity differences between the air streams passing through the primary and secondary lumens to reduce the sound output of the aspiration device. In some embodiments, the meeting point of the two air streams can be altered in a desired manner, while in other embodiments the relative speeds of one or more of the streams can be altered relative to the other.

In various embodiments, the handle can have an optional an ergonomic hourglass-like shape, if desired. In some embodiments, one or more textured or knurled surfaces can be formed on the outside surfaces of the handle for user convenience. In other embodiments, the aspirator tube/tip many have an angle fabricated therein, or the handle may have a curved or angled primary lumen. The aspirator tip may optionally have holes near a distal end of the tube/tip.

In one exemplary embodiment, the handle of the present invention may be fabricated out of a polymeric or plastic material such as Delrin acetate, or any other medical or dental material which has good sound dampening properties. Alternatively, the device may be fabricated out of metal or other materials.

FIG. 3D depicts various exemplary dimensions of the embodiment of FIG. 2A, which can desirably result in reduced sound levels as disclosed herein. If desired, alternative handle designs can incorporate larger and/or smaller size components while maintaining equivalent dimension rations between the various components disclosed herein.

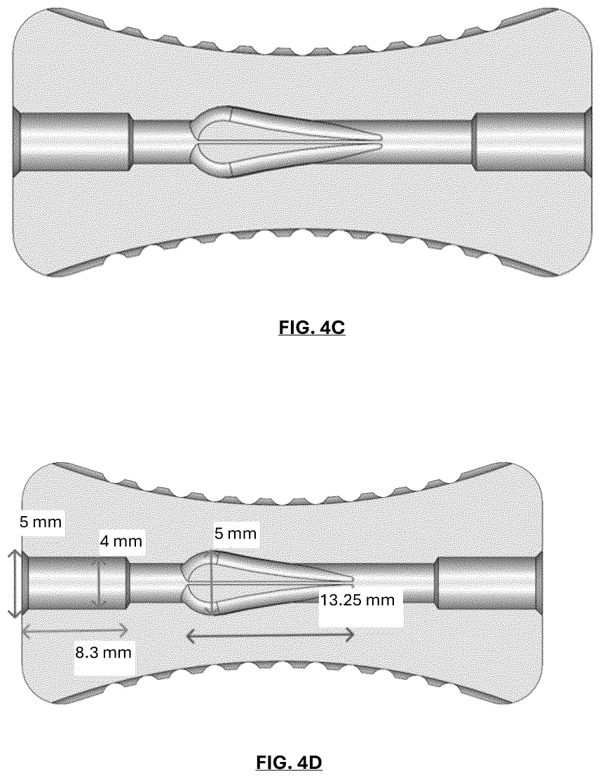

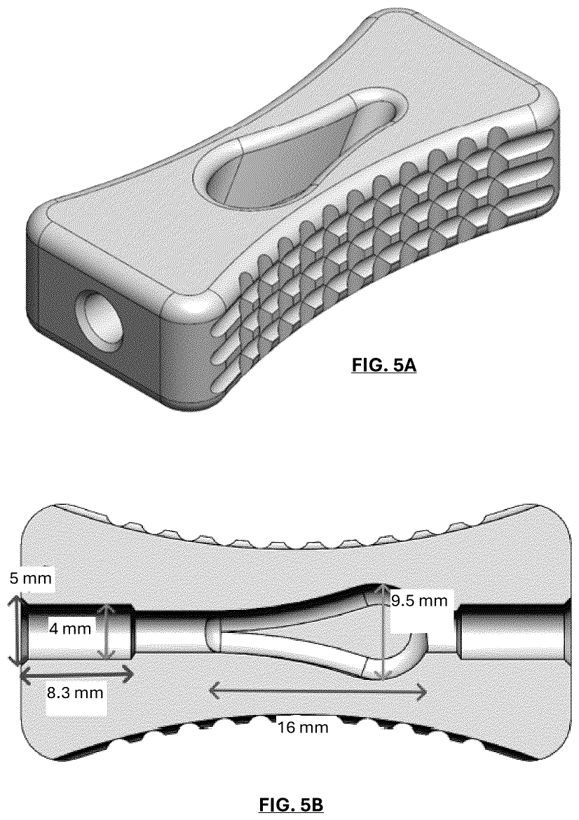

FIGS. 4A through 4D depict various views of one alternative embodiment of a noise-reducing handle for an aspirator tool, with FIG. 4D depicting various exemplary dimensions thereof. As previously noted, alternative handle designs can incorporate larger and/or smaller size components while maintaining equivalent dimension rations between the various components disclosed herein.

FIGS. 5A and 5B depict views of another alternative embodiment of a noise-reducing handle for an aspirator tool, with FIG. 5B depicting various exemplary dimensions thereof. As previously noted, alternative handle designs can incorporate larger and/or smaller size components while maintaining equivalent dimension rations between the various components disclosed herein.

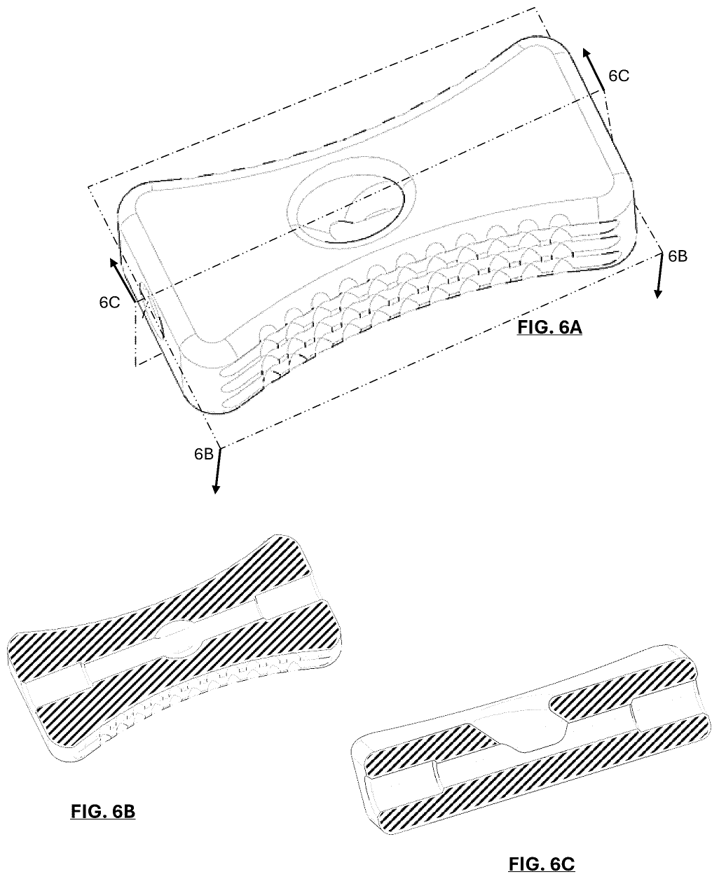

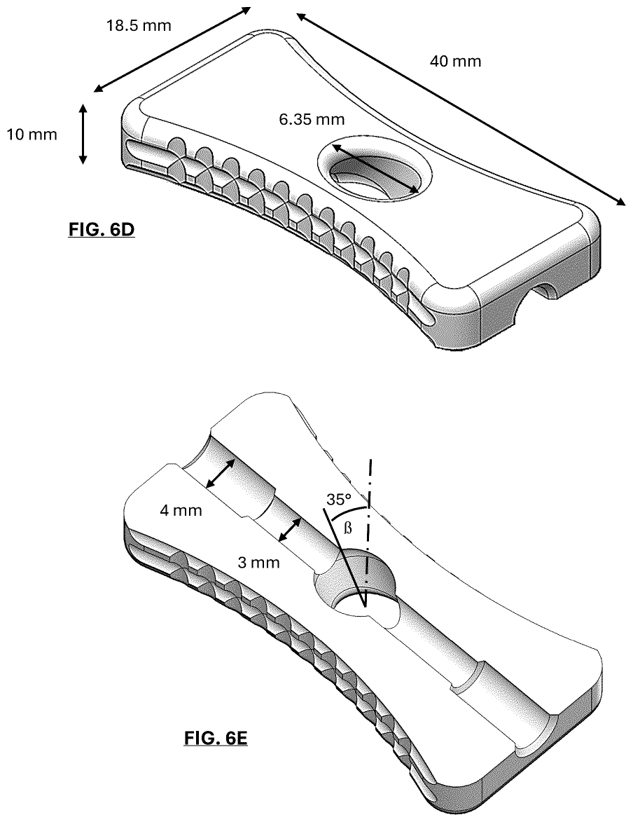

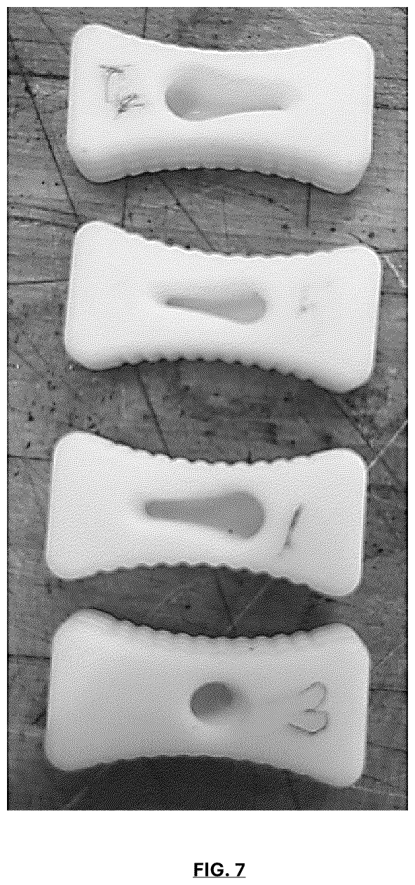

FIGS. 6A through 6E depict various views of another alternative embodiment of a noise-reducing handle for an aspirator tool which demonstrated superior sound reduction measurements during various tests.

FIG. 7 depicts a view of additional alterative handle configurations constructed in accordance with the teachings of the present invention.

In the embodiments disclosed herein, it should be understood that, in some other alternative embodiments, the arrangement of the inlet and outlet openings might be reversible (e.g., the “handle” can be turned around, with the outlet being used as an inlet, and the inlet being used as the outlet), which use can be facilitated where the inlet and outlet openings and/or lumens thereof are of a similar size and/or shape (although different sized/shaped outlet and inlets on a single housing are also contemplated in the various embodiments herein). Where the handle might optionally be reversed in such a manner, it can allow physician or technician to “test” the sound level of a given aspiration machine with the handle in a first direction and then test again with the handle in a second, reverse direction, with the handle utilized in whichever direction is most advantageous. Because generated sound levels can change with the type, model, settings and attachments of a given suction machine, this reversibility of the handle can allow for additional sound reductions across various environments and equipment.

The present invention provides various components, devices, systems and methods for treating various anatomical structures of the human and/or animal anatomy.

In accordance with another aspect of the present subject matter, various methods for manufacturing and/or assembling a handle as set for within any of the details described with the present application are provided.

If desired, an aspirator handle constructed in accordance with the various teaching herein could be constructed from a variety of modular components, including modular components comprising different materials. If desired, such modular components could be provided in a kit form for selection and/or assembly in a surgical theatre and/or in situ during a surgical procedure. If desired, various components described herein may be removable and replaceable.

While embodiments and applications of the present subject matter have been shown and described, it would be apparent that other embodiments, applications and aspects are possible and are thus contemplated and are within the scope of this application. The subject matter, therefore, is not to be restricted except in the spirit of the appended claims.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

As previously noted, the use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A suction device for an aspirator connectable to a vacuum source, comprising:
a body having a first end having an inlet port, a second end having an outlet port, and a primary lumen extending through the body from the inlet port to the outlet port;
an air chamber positioned within the primary lumen; and
a control opening laterally spaced from the primary lumen, the control opening in fluid communication with the air chamber through a secondary lumen;
the primary lumen having a first diameter proximate to the inlet port and a second diameter proximate to the air chamber, the first diameter being larger than the second diameter.

2. The suction device of claim 1, wherein the secondary lumen intersects the primary lumen at an obtuse angle.

3. The suction device of claim 1, wherein the primary lumen has a third diameter proximate to the outlet port, and the third diameter is larger than the second diameter.

4. The suction device of claim 1, wherein the control opening is positioned on a side surface of the body.

5. The suction device of claim 4, wherein the control opening can be selectively occluded by a user of the aspirator.

6. The suction device of claim 1, wherein the control opening is oblong.

7. The suction device of claim 1, wherein the control opening is round.

8. The suction device of claim 1, wherein the control opening is teardrop shaped.

9. The suction device of claim 1, wherein the air chamber control is spherical.

10. The suction device of claim 1, wherein the air chamber control is teardrop shaped.

11. The suction device of claim 1, wherein the handle is reversible.
